(19) United States
(12) Patent Application Publication
(10) Pub. No.: US 2024/0018100 A1
(43) Pub. Date: Jan. 18, 2024

(54) METHOD FOR PREPARING METHYL MERCAPTAN WITH TREATMENT OF GASEOUS WASTE

(71) Applicant: ARKEMA FRANCE, Colombes (FR)

(72) Inventors: Georges FREMY, Lacq (FR); Jean-Michel RAYMOND, Cauneille (FR); Eric LAMANT, Lacq (FR)

(73) Assignee: ARKEMA FRANCE, Colombes (FR)

(21) Appl. No.: 18/256,703

(22) PCT Filed: Dec. 16, 2021
(86) PCT No.: PCT/FR2021/052365

§ 371 Date: Jun. 9, 2023

(30) Foreign Application Priority Data
Dec. 17, 2020 (FR) 2013438

(51) Int. Cl. C07C 319/08; C07C 319/28

(52) U.S. Cl. CPC C07C 319/08; C07C 319/28

(57) ABSTRACT

The present invention relates to a process for producing methyl mercaptan, comprising the following steps:
A) methanol is reacted with hydrogen sulfide to form a stream (M), preferably in gaseous form, comprising methyl mercaptan, unreacted H₂S and possibly sulfur byproducts;
B) optionally, said stream (M) is condensed;
C) at least one step of purification of said stream (M) is performed to obtain a stream enriched in methyl mercaptan;
D) the gaseous vents resulting from said at least one purification step are recovered, said gaseous vents comprising at least one sulfur compound, preferably H₂S;
E) a gas-liquid extraction of said at least one sulfur compound, preferably H₂S, is performed with liquid methanol so as to obtain a liquid methanol enriched in sulfur compound(s), preferably in H₂S; and
F) optionally, said enriched methanol is used as reagent for the reaction of step A).

The present invention relates to a process for producing methyl mercaptan incorporating treatment of the gaseous discharges. The present invention also relates to a process for treating the gaseous discharges from a methyl mercaptan production unit.

Mercaptans are of great interest industrially and are currently in widespread use in the chemical industries, notably as starting materials in the synthesis of more complex organic molecules. For example, methyl mercaptan (CH₃SH or MeSH) is used as a starting material in the synthesis of methionine, an essential amino acid for animal nutrition. Methyl mercaptan is also used in the synthesis of dialkyl disulfides, in particular in the synthesis of dimethyl disulfide (DMDS), a sulfiding additive for hydrotreating catalysts for petroleum fractions, among other applications.

The industrial synthesis of methyl mercaptan may take place according to “the methanol route”, starting with methanol and hydrogen sulfide according to the following reaction (1):

CH₃OH+H₂S→CH₃SH+H₂O (1)

Reaction (1) is usually performed in the gas phase with H₂S in excess. Also, at the industrial level, gaseous discharges (also called gaseous vents) may be emitted throughout the production process. These discharges are usually treated by incineration. However, they include a very high concentration of H₂S whose combustion during incineration will lead to high emissions of sulfur dioxide into the atmosphere (SO₂ is a pollutant and irritant gas, which can be responsible for acid rain). Furthermore, this loss of H₂S increases the variable production costs of methyl mercaptan and leads to a decrease in productivity.

There is thus a need for a process for producing methyl mercaptan which is more environmentally friendly and more economical. There is also a need to reduce the gaseous discharges, and notably the sulfur dioxide, emitted during the production of methyl mercaptan.

One object of the present invention is to provide a process for preparing methyl mercaptan which allows improved management of the gaseous discharges, and which is notably more environmentally friendly.

One object of the present invention is to reduce the gaseous discharges, and notably the amount of sulfur dioxide, emitted during the production of methyl mercaptan.

Another object of the present invention is to provide a process for preparing methyl mercaptan, which is more economical.

Another object of the present invention is to provide a process and/or a device for treating gaseous discharges, which can be readily integrated into a unit for producing methyl mercaptan, notably via the methanol route.

The present invention meets all or some of the abovementioned objectives.

The present inventors have discovered, surprisingly, that the gaseous discharges can be recovered and treated by gas-liquid extraction. The gas(vent gases)-liquid(methanol) extraction according to the invention makes it possible notably to pass the sulfur compounds contained in said gaseous vents into the liquid methanol. The term “sulfur compounds” means compounds which comprise at least one sulfur atom, preferably 1 or 2 sulfur atoms. In particular, the term “sulfur compounds” means H₂S, methyl mercaptan and sulfur byproducts such as dimethyl sulfide (DMS) and dimethyl disulfide (DMDS).

Thus, the gas-liquid extraction according to the invention makes it possible to recover at least one sulfur compound, preferably H₂S, in methanol. In particular, the gas-liquid extraction according to the invention makes it possible to recover the H₂S and methyl mercaptan in methanol.

Very advantageously, said methanol enriched in sulfur compound(s) can be used, preferably used directly (for example without a purification step), as a reagent for forming methyl mercaptan. Thus, the present invention makes it possible to reintroduce into the production process the H₂S and possibly the methyl mercaptan from the vent gases which were hitherto incinerated.

Furthermore, said extraction allows the inert compounds to pass (i.e. the inert compounds hardly pass from the vent gases into the methanol but remain in the gas phase). This avoids their accumulation in the installations and thus blockages and the resulting safety problems. In particular, hydrogen (H₂), does not pass into the methanol, which avoids undesirable methanation reactions in the reactor when enriched methanol is used as a reagent in the production of methyl mercaptan. These reactions are notably the following:

CH₃OH+H₂→CH₄+H₂O and CH₃SH+H₂→CH₄+H₂S.

It is understood that the extraction according to the invention is not intended to recover the sulfur compounds, and in particular the unreacted H₂S, directly from the outlet of the reactor for the production of methyl mercaptan from methanol and H₂S. The extraction according to the invention is also not aimed at the recovery or recycling of the majority of the unreacted H₂S. The process according to the invention is directed toward recovering the sulfur compounds present in the gaseous vents, which are usually incinerated and responsible for the release of sulfur dioxide.

Thus, the process according to the invention notably does not involve an amount of methanol for said extraction greater than the amount of methanol required for the synthesis of the methyl mercaptan. On the contrary, the extraction according to the invention can easily be integrated into a methyl mercaptan production unit since it treats only the gaseous vents. It therefore consumes little energy and uses a simple device.

Said extraction also makes it possible to reduce the amount of vent gases to be treated by incineration and makes it possible to greatly reduce the release of sulfur dioxide into the atmosphere.

The process for producing methyl mercaptan according to the invention is thus more economical and has better productivity, while at the same time being more environmentally friendly.

The present invention thus relates to a process for producing methyl mercaptan, comprising the following steps:
A) methanol is reacted with hydrogen sulfide to form a stream (M), preferably in gaseous form, comprising methyl mercaptan, unreacted H₂S and possibly sulfur byproducts;
B) optionally, said stream (M) is condensed;
C) at least one step of purification of said stream (M) is performed to obtain a stream enriched in methyl mercaptan;
D) the gaseous vents resulting from said at least one purification step are recovered, said gaseous vents comprising at least one sulfur compound, preferably H₂S;
E) a gas-liquid extraction of said at least one sulfur compound (present in said vent gases), preferably H₂S, is performed with liquid methanol so as to obtain a liquid methanol enriched in sulfur compound(s), preferably in H₂S; and
F) optionally, said enriched methanol is used as reagent for the reaction of step A).

In particular, the gaseous vents comprise H₂S, methyl mercaptan and possibly sulfur byproducts so as to obtain, following step E), a methanol enriched in H₂S, methyl mercaptan and possibly sulfur byproducts.

The term “enriched methanol” means the methanol obtained after the gas-liquid extraction according to the invention, notably obtained after step E).

In particular, the enriched methanol is a composition comprising methanol and at least one sulfur compound, preferably comprising methanol and H₂S, optionally methyl mercaptan and optionally sulfur byproducts such as DMS and DMDS. In particular, said enriched methanol comprises between 0.1% and 20% by weight of H₂S, preferably between 1% and 10%, more preferentially between 1% and 5% by weight of H₂S, relative to the total weight of the enriched methanol.

More particularly, the term “enriched methanol” means a composition comprising:
methanol, preferably at least 50% by weight of methanol, more preferentially at least 80% by weight, more preferentially at least 90% by weight of methanol, relative to the total weight of the composition;
H₂S, preferably between 0.1% and 20% by weight of H₂S, more preferentially between 1% and 10%, even more preferentially between 1% and 5% by weight of H₂S, relative to the total weight of the composition;
optionally methyl mercaptan;
optionally sulfur byproducts, preferably dimethyl sulfide and dimethyl disulfide;
optionally water; and
optionally inert compounds.

Said composition may thus comprise:
between 90% and 99% by weight of methanol;
between 0.1% and 10%, preferably between 0.1% and 5% by weight of H₂S; and
between 0.1% and 5% by weight of methyl mercaptan, relative to the total weight of the composition.

The term “vent gas or gaseous discharge” means a gaseous phase comprising at least one sulfur compound as defined above and notably recovered following at least one step of purification of stream (M). Said gaseous vents may comprise, or even consist of, H₂S, methyl mercaptan, inert compounds, possibly water and sulfur byproducts. They comprise in particular at least 50%, preferably at least 60%, or even at least 70% by weight of H₂S relative to the total weight of the vent gases. They may comprise:
between 50% and 90%, preferably between 60% and 80% by weight of H₂S;
between 5% and 25%, preferably between 10% and 20% by weight of methyl mercaptan;
between 1% and 15% of inert matter, preferably between 5% and 15% of inert matter;
relative to the total weight of the gaseous vents, and possibly of the water and trace sulfur byproducts.

In particular, the gaseous vents according to the invention are normally considered as waste or rejects and are usually sent to the incinerator.

In particular, the gaseous vents for the purposes of the present invention are not recovered directly at the outlet of the reactor in which step A) takes place.

Said vent gases may come from a decanter and/or from a purge, preferably from a purge of a gas stream.

The term “inert matter” or “inert compounds” means compounds which are not chemically active during the preparation of methyl mercaptan from methanol and H₂S. Inert compounds that may be mentioned include CH₄, CO, CO₂, H₂ and N₂.

The term “traces” of a compound means an amount of between 0 and 10 000 ppm, preferably between 0 and 1000 ppm.

The term “methyl mercaptan purification step” notably means a step for producing a stream enriched in methyl mercaptan. The term “stream enriched in methyl mercaptan” notably means a stream which comprises a weight percentage of methyl mercaptan (relative to the total weight of said stream) greater than the weight percentage of methyl mercaptan relative to the total weight of said stream before said purification step.

According to the present invention, the unit ppm (part per million) refers to a mass fraction.

Step A)—Reaction:

In step A), methanol is reacted with hydrogen sulfide to form a stream (M), preferably in gaseous form, comprising methyl mercaptan, unreacted H₂S and possibly sulfur byproducts. Stream (M) may also contain water and unreacted methanol.

Prior to step A), a gaseous stream of the H₂S and methanol reagents may be prepared as follows.

Liquid methanol is injected into gaseous H₂S. This injection enables the methanol to be partially or totally vaporized. The mixture of H₂S and methanol can then be totally vaporized, if necessary, so as to obtain a totally gaseous stream.

Thus, a gas stream of H₂S and methanol, preferably prepared as above, or separately methanol and H₂S, each in gaseous form, are introduced into a reactor.

Said reactor may be isothermal or adiabatic, with plates, multi-tubular or with a fixed bed. An adiabatic reactor is preferably chosen.

The reaction temperature may be between 200° C. and 500° C., preferably between 200° C. and 400° C. Preferably, the reaction temperature is between 200° C. and 360° C. Above this temperature, the catalyst may be physically damaged (notably by sintering and coking).

The pressure may be between 1 and 40 bar.

The H₂S/methanol mole ratio may be between 1 and 50, preferably between 1 and 25. The H₂S is preferably in excess relative to methanol.

The reactor may contain a catalyst for the methyl mercaptan formation reaction, preferably in the gas phase. Among the catalysts that may be used, mention may be made of:
alumina-based catalysts;
thorium dioxide ThO₂, preferably deposited on a silicate support;
catalysts based on cadmium sulfide, preferably on an alumina support;
catalysts based on the following oxides: MgO, ZrO₂, rutile (R) and anatase (A) TiO₂, CeO₂, and γ-Al₂O₃;
catalysts based on metal oxides, preferably doped with alkali metals (Li, Na, K, Rb, Cs) and optionally supported on SiO₂, Al₂O₃ or Nb₂O₅;
catalysts based on alkali metal carbonates;
catalysts based on alkali metal salts with certain acids of transition metals (Cr, Mo, W, Ni), impregnated on γ-alumina or other metal oxides;
potassium tungstate on alumina K₂WO₄/Al₂O₃.

A stream (M) is thus obtained comprising methyl mercaptan, unreacted H₂S and possibly sulfur byproducts.

### Step B)—Condensation:

Stream (M) obtained on conclusion of step A) may optionally be condensed by means of any conventional technique, preferably using one or more condensers or economizers. During condensation, stream (M) is notably cooled as low as possible, to maximize the removal of water, but must be maintained strictly above 16° C. to avoid the formation of solid hydrates of methyl mercaptan. Preferably, stream (M) is condensed at a temperature of between 20° C. and 70° C., for example between 30° C. and 60° C.

### Step C)—Purification and Step D)—Recovery of the Vent Gases:

Preferably, in step C), said at least one purification step corresponds to at least one step of phase separation, preferably by decantation, and/or to at least one distillation step. Step C) may notably correspond to one or more phase separation steps, for example one or two decantation steps, and/or one or more distillation steps, for example one or two distillation steps.

Preferably, the gaseous vents are recovered following at least one phase separation step, in particular by decantation, and/or by performing at least one purge, preferably a purge of a gas stream (for example of a gas stream comprising at least one sulfur compound). Said purge can be performed after a phase separation step, on the gas stream obtained (for example on a gas stream comprising at least one sulfur compound). In particular, the decantation step makes it possible to separate an aqueous stream from an organic stream comprising methyl mercaptan and optionally a gas stream comprising at least one sulfur compound; said vent gases emitted during decantation are then recovered. They may be vent gases recovered from the decanter or vent gases recovered in one of the phases separated, for example by stripping with inert gas or by thermal stripping.

The following may thus be separated out, preferably by decantation, from stream (M):
a gas stream comprising unreacted hydrogen sulfide, said stream being purged so as to recover a gaseous vent; and
a stream comprising methyl mercaptan, preferably in liquid form.

Preferably, step C) makes it possible, via one or more purification steps, to remove from stream (M) the unreacted H₂S and/or the sulfur byproducts and/or the water. In particular, after step C), a stream enriched in methyl mercaptan is obtained.

In particular, the gaseous vents E1 or E1′ and/or E2 and/or E3 are obtained and recovered according to the invention as defined below. They may or may not be combined before performing the gas-liquid extraction.

The purification step C) may be performed via any conventional technique and in particular according to steps c1) to c4) as described below.

Thus, according to one embodiment, step C) comprises the following purification steps:
c1) the following are separated out, preferably by decantation, from stream (M):
   a gas stream (N) comprising unreacted hydrogen sulfide, said stream (N) being purged so as to recover a gaseous vent E1;
   an aqueous stream (O); and
   a stream (P) comprising methyl mercaptan, unreacted hydrogen sulfide, water and sulfur byproducts;
c2) stream (P) is distilled so as to obtain:
   a stream (R) comprising hydrogen sulfide, preferably at the top of the column; and
   a stream (S) comprising methyl mercaptan, water and sulfur byproducts, preferably at the bottom of the column;
c3) stream (S) is distilled so as to obtain:
   a stream (T) comprising methyl mercaptan and water, preferably at the top of the column; and
   a stream (U) comprising the sulfur byproducts, preferably at the bottom of the column;
c4) the methyl mercaptan and the water are separated, preferably by decantation, so as to obtain:
   a stream (V) comprising methyl mercaptan and water; and
   a stream (W) comprising water; and
   a gaseous vent E2.

The vent gases E1 and/or E2 can be sent to the methanol absorber (defined below). Said streams (M) and/or (P) and/or (S) and/or (T) and/or (V) may possibly comprise unreacted methanol and water, the methanol preferably being in trace amounts.

Step c1—Separation:

The separation step c1), preferably by decantation, produces:
a gas stream (N) comprising unreacted hydrogen sulfide;
an aqueous stream (O); and
a stream (P) comprising methyl mercaptan, water, unreacted hydrogen sulfide and sulfur byproducts.

Preferably, stream (M) is separated at a temperature of between 20° C. and 70° C., preferably between 30° C. and 60° C. The pressure may be between 1 and 40 bar absolute.

Stream (P) obtained may notably be in gaseous form or in liquid form. When stream (P) is in gaseous form, streams (N) and (P) may be combined.

In particular, the aqueous stream (O), preferably in liquid form, comprises at least 50%, preferably at least 70%, more preferentially at least 90% by weight of water, relative to the total weight of the water present in stream (M). The aqueous stream (O) may thus be sent to a degasser. The degassed aqueous stream may then be sent for waste water treatment.

The gas stream (N) can be recycled into the reactor feed for step A) and/or purging of this stream (N) can be performed so as to avoid the accumulation of inert matter and/or impurities. Examples of inert matter and/or impurities that may be mentioned include: methane, CO, CO₂, H₂ and N₂. The gas stream resulting from this purging is called the vent gases E1. When streams (N) and (P) are combined, the same type of purging may be performed so as to obtain a gas stream called the vent gases E1′.

The vent gases E1 or E1′ can be sent to the methanol absorber.

Step c2—Removal of H₂S by Distillation:

Distillation of stream (P) is then be performed so as to obtain:
a stream (R) comprising hydrogen sulfide, preferably at the top of the column; and
a stream (S) comprising methyl mercaptan, water and sulfur byproducts, preferably at the bottom of the column;

During the distillation, the pressure may be between 1 and 40 bar absolute, and/or the temperature may be between −60° C. and +60° C., at the top of the column, and between +20° C. and +200° C. at the bottom of the column.

Stream (R) comprising H₂S may be recovered at the top of the column, and optionally recycled into the reactor feed for step A).

In particular, said distillation of step c2) makes it possible to remove the H₂S remaining in stream (P) (it is understood that traces of H₂S may remain in stream (S)).

Step c3—Removal of the Sulfur Byproducts by Distillation:

Distillation of stream (S) is performed so as to obtain:
a stream (T) comprising methyl mercaptan and water, preferably at the top of the column; and
a stream (U) comprising the sulfur byproducts, preferably at the bottom of the column.

During the distillation, the pressure may be between 1 and 40 bar absolute, and/or the temperature may be between +20° C. and +100° C., at the top of the column, and between +40° C. and +200° C. at the bottom of the column.

In particular, said distillation of step c3) makes it possible to remove the sulfur byproducts remaining in stream (S) (it is understood that traces of the sulfur byproducts may remain in stream (T)).

Step c4—Separation of Methyl Mercaptan and Water:

Prior to step c4), stream (T) can be cooled as low as possible, to maximize the water removal, but must be kept strictly above 16° C. to avoid the formation of solid hydrates of methyl mercaptan. Preferably, stream (T) is cooled to a temperature of between 20° C. and 70° C., for example between 30° C. and 60° C.

This cooling makes it possible to maximize the separation of water during step c4), while maintaining a temperature strictly above 16° C. to avoid the formation of solid hydrates of methyl mercaptan. Separation of the methyl mercaptan and of the remaining water can then be performed, preferably by decantation, so as to obtain:
a stream (V) comprising methyl mercaptan and water, preferably in liquid form;
a stream (W) comprising water, preferably in liquid form; and
a vent gas E2.

In particular, in step c4), stream (W) comprises at least 50% by weight, preferably at least 70%, more preferentially at least 90% by weight of water, relative to the total weight of the water present in stream (T).

In the separation step c4), it is possible to recover the gas phase thus separated from streams (W) and (V), which are both in liquid form. This gaseous discharge is called the vent gases E2.

The vent gases E2 can be sent to the methanol absorber.

Stream (V) or stream (T) obtained can then be dried according to the drying process as described below.

Step c5)—Drying:

According to one embodiment, stream (V) obtained following steps c1) to c4) is dried according to the drying process c5). According to another embodiment, the drying process c5) is performed on any stream comprising methyl mercaptan and water.

Said process c5) for drying methyl mercaptan comprises the following steps:
1′) a stream (A) comprising methyl mercaptan and water is introduced into a distillation column (1);
2′) said stream (A) is distilled in said column (1);
3′) the distillate (B) is recovered in gaseous form, preferably at the top of the column;
4′) the distillate (B) is condensed, preferably in a condenser (2), so as to obtain a condensate (C) in liquid form;
5′) said condensate (C) is separated, preferably using a decanter (3), so as to obtain two separate liquid phases:
   an aqueous phase (D); and
   an organic phase (E) comprising methyl mercaptan;
6′) all or part of the organic phase (E) is optionally introduced into the distillation column (1) as reflux; and
7′) a stream (F) comprising the dried methyl mercaptan is optionally recovered, preferably at the bottom of column (1); and
8′) the vent gas E3 of said aqueous phase (D) is recovered, preferably after decantation.

In particular, the term “dried methyl mercaptan” means a methyl mercaptan comprising between 0 and 1500 ppm, preferably between 0 and 1000 ppm, for example between 10 and 800 ppm, more preferentially between 40 and 800 ppm of water, relative to the total by weight of methyl mercaptan and water. It is recovered from the distillation column, preferably at the bottom of the distillation column.

The distillation of step 2′) may be performed at a pressure of between 0.05 and 75 bar absolute, preferably between 1 and 30 bar absolute, more preferentially between 5 and 15 bar absolute, for example at about 10, 11, 12, 13, 14 or 15 bar absolute.

The distillation of step 2′) may be performed at a temperature of between 20° C. and 200° C., preferably between 60° C. and 100° C., more preferentially between 65° C. and 95° C.; for example between 70° C. and 90° C. Preferably, the distillation of step 2) may be performed at a temperature of between 40° C. and 200° C., preferably between 80° C. and 100° C. at the bottom of the column, and between 20° C. and 100° C., preferably between 60° C. and 80° C. at the top of the column.

Particularly preferably, the distillation of step 2′) is performed at a pressure of between 5 and 15 bar absolute and at a temperature of between 60° C. and 100° C. In particular, the distillation of step 2′) is performed at a pressure of between 5 and 15 bar absolute and at a temperature of between 70° C. and 90° C. In particular, the distillation of step 2′) is an azeotropic distillation.

The distillation of step 2′) may be performed in any known type of distillation column. It may be a column with plates (for example plates with caps, plates with valves or perforated plates) or with packing (for example with bulk or structured packing). The distillation of step 2′) may be performed in a plate column, preferably comprising between 5 and 50 plates, more preferentially between 10 and 40 plates, for example between 25 and 30 plates. The distillation of step 2′) may also be performed in a partition column (“DWC” or Divided Wall Column). The partition may be fixed or mobile, for example with structured or bulk packing.

Stream (A) is preferably in liquid or gaseous form.

Preferably, stream (A) comprises, or even consists of, methyl mercaptan, water and possibly traces of methanol, H₂S and sulfur byproducts.

Stream (A) may comprise at least 90%, preferably at least 95%, more preferentially at least 98%, for example at least 99% by weight of methyl mercaptan, relative to the total by weight of methyl mercaptan and water.

Stream (A) may comprise at least 0.15% by weight of water, preferably at least strictly greater than 0.15% by weight of water, relative to the total weight of water and methyl mercaptan. Stream (A) may comprise a maximum of 30%, preferably a maximum of 10% by weight of water, relative to the total by weight of methyl mercaptan and water.

Stream (A) may comprise between 0.15%, preferably strictly greater than 0.15%, and 30% by weight of water relative to the total by weight of methyl mercaptan and water.

Stream (A) may comprise between 0.15%, preferably strictly greater than 0.15%, and 10% by weight of water relative to the total by weight of methyl mercaptan and water.

Preferably, stream (A) comprises between 0.15%, preferably strictly greater than 0.15%, and 5% by weight of water relative to the total by weight of methyl mercaptan and water.

For example, stream (A) comprises between 0.15%, preferably strictly greater than 0.15%, and 2%, for example between 0.15% and 1.5% or between 0.15% and 1% in weight of water, relative to the total by weight of methyl mercaptan and water; the remainder possibly being methyl mercaptan.

Following step 2′) of distillation of stream (A), a gaseous distillate (B) is obtained. This distillate (B) corresponds notably to an azeotropic mixture, preferably a heteroazeotropic mixture, in particular under the pressure and/or temperature conditions of distillation step 2′).

Thus, the distillation of step 2′) makes it possible notably to form an azeotropic mixture (i.e. an azeotropic distillation). Once recovered and condensed in liquid form (condensate (C)), it is found in two-phase form, the two phases of which can be readily separated, notably by decantation.

Step 4′) of condensation of the distillate (B) may be performed via any conventional technique. The condensation may be performed in a condenser separate from the distillation column or which can be integrated into said column. A condensate (C) in liquid form is then obtained, preferably comprising two phases, one of which is aqueous and the other organic (and comprising the methyl mercaptan). During the condensation step 4′), the temperature may be between 20° C. and 50° C. and/or the pressure may be between 5 and 15 bar absolute.

The distillate (B) and the condensate (C) preferably have the same composition. During step 5′) of separation, any known method may be used. Most preferably, decantation is used. During the separation step, the temperature may be between 20° C. and 50° C. and/or the pressure may be between 5 and 15 bar absolute. On conclusion of step 5′), two separate liquid phases are obtained:
an aqueous phase (D); and
an organic phase (E) comprising methyl mercaptan.

According to one embodiment, the aqueous phase (D) comprises:
water,
H₂S, preferably in trace amounts,
possibly methyl mercaptan, preferably in trace amounts; and
possibly sulfur byproducts, preferably in trace amounts.

The H₂S, and possibly methyl mercaptan and sulfur byproducts, are preferably dissolved in said aqueous phase. They may be separated from this aqueous phase via any known means and preferably by stripping, which may be thermal stripping or by stripping with inert gas (for example by stripping with nitrogen, methane or CO₂). A gaseous phase is obtained, which then forms vent gases called the vent gases E3 below. The vent gases E3 can be sent to the methanol absorber.

According to one embodiment, the organic phase (E) is recovered on conclusion of step 5′) when the reflux step 6′) is not performed. According to another embodiment, the organic phase (E) is used totally or partly as reflux for the distillation column (1).

In step 6′), the reflux ratio may be between 0 and 0.99, preferably between 0 and 0.60. The term “reflux ratio” means the mass ratio [organic phase (E)/stream (A)].

The drying process may be performed continuously or batchwise, preferably continuously.

During steps 1′) to 7′) of the process, the pressure may be between 0.05 and 75 bar absolute, preferably between 1 and 30 bar absolute, more preferentially between 5 and 15 bar absolute, for example approximately 10, 11, 12, 13, 14 or 15 bar absolute. Methanol, preferably in trace amounts, may be included in stream (A) and/or the distillate (B) and/or the condensate (C) and/or the aqueous phase (D) and/or stream (F).

### Step E)—Gas-Liquid Extraction:

The gas-liquid extraction can be performed in at least one absorption column or in at least one tank, preferably with mechanical stirring. Said absorption column(s) are notably chosen from packed columns (for example with bulk or structured packing), bubble columns, spray columns and falling film columns. Preferably, one or more packed columns are used, for example between 1 and 10 columns. Several absorption columns may be used, in parallel or in series.

The flow rates of the gas (vent gases) and liquid (methanol) phases depend on the type and number of columns. The gaseous vents and the liquid methanol arrive in the absorption column(s) co-currentwise or counter-currentwise, preferably counter-currentwise. For example, the gaseous vents arrive from the bottom of the column(s) and the liquid methanol from the top of the column(s).

This type of device for performing a gas-liquid extraction is generally called an “absorber”, and in the case of the present invention, a “methanol absorber”.

The gas-liquid extraction may be performed at a temperature of between 0° C. and 80° C., for example between 5° C. and 80° C., preferably between 10° C. and 80° C., more preferentially between 20° C. and 70° C. The gas-liquid extraction is performed at a pressure of between 4 and 60 bar absolute, preferably between 10 and 50 bar absolute.

The mass ratio of the gaseous vents relative to the methanol may be between 0.001 and 0.5, preferably between 0.005 and 0.1.

Step E) notably makes it possible to pass the sulfur compounds into the liquid methanol and to reduce, or even avoid, the discharge of SO₂ into the atmosphere.

The gaseous vents thus treated (that is to say of which at least one sulfur compound has been absorbed in the methanol) can then be recovered, possibly incinerated, and released into the atmosphere with a reduced content of SO₂; preferably, said vent gases comprise almost no or no SO₂.

### Step F)—Recycling:

Preferably, the enriched methanol thus obtained is used as reagent for the reaction of step A), optionally as a mixture with fresh methanol.

For the purposes of the present invention, the term “fresh methanol” means a non-enriched methanol, i.e. a methanol which has not undergone the gas-liquid extraction according to the invention.

The present invention also relates to a process for treating the gaseous discharges emitted by a unit for producing methyl mercaptan from methanol and H₂S, comprising the following steps:
recovery of the gaseous vents from at least one methyl mercaptan purification step, said gaseous vents comprising at least one sulfur compound, preferably H₂S;
performing a gas-liquid extraction of said at least one sulfur compound, preferably H₂S, with liquid methanol so as to obtain a liquid methanol enriched in sulfur compound(s), preferably in H₂S; and
optionally using said enriched methanol as reagent for the reaction for the production of methyl mercaptan from methanol and H₂S.

All the elements of the process for treating gaseous discharges (notably the reaction for producing methyl mercaptan, said at least one purification step, said gaseous vents, said at least one sulfur compound and said gas-liquid extraction) are as defined for the process for producing methyl mercaptan according to the invention.

### DESCRIPTION OF THE FIGURES

FIG. 1 shows an embodiment of a process for producing methyl mercaptan via the methanol route in which the vent gases E1 and E2 are recovered.

The reaction step A) is performed in a reactor (1) using methanol and H₂S.

Stream (M) leaving the reactor (1) comprises MeSH, water, H₂S and sulfur byproducts.

Stream (M) is condensed in a condenser (II). It is then separated in a decanter (Ill) into three streams:

a stream (N) comprising H₂S;

a stream (O) comprising water; and

a stream (P) comprising MeSH, water, H₂S and sulfur byproducts.

Stream (N) is purged and said purge represents the vent gas E1.

Stream (P) is distilled in a distillation column (IV) to remove the H₂S (stream (R) at the top of the column) and to obtain a stream (S) at the bottom of the column comprising MeSH, water and sulfur byproducts. Stream (S) is then distilled in a distillation column (V) to obtain a stream (U) at the bottom of the column comprising the sulfur byproducts and a stream (T) at the top of the column comprising the MeSH and the water. Stream (T) is then separated in a decanter (VI) into a stream (V) comprising MeSH and water and a stream (W) comprising water. The vent gas from this decanter is recovered and represents the vent gas E2.

FIG. 2 shows one embodiment of the drying process in which the vent gas E3 is recovered.

Stream (A) enters distillation column (1). Stream (A) is distilled in column (1). The distillate (B) is recovered at the top of the column in gaseous form. The distillate (B) is then condensed in a condenser (2) where it is recovered in two-phase liquid form (condensate (C)). The condensate (C) then settles in the decanter (3) so as to obtain:

an aqueous phase (D); and

an organic phase (E).

The organic phase (E) then serves as reflux for the distillation column (1).

After decantation, the vent gas E3 is recovered by stripping with an inert gas from the aqueous phase (D).

The dried methyl mercaptan is recovered at the bottom of column (1) (stream (F)).

The expression “between X and X” includes the limits mentioned, unless mentioned otherwise.

The examples that follow illustrate the present invention but are not in any way limiting.

### EXAMPLES

### Example 1: Comparative Example, without Gas-Liquid Extraction

The conditions are as follows:

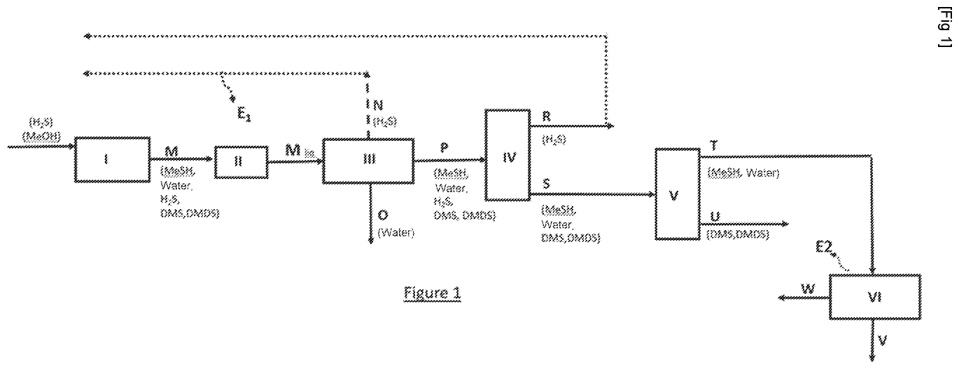

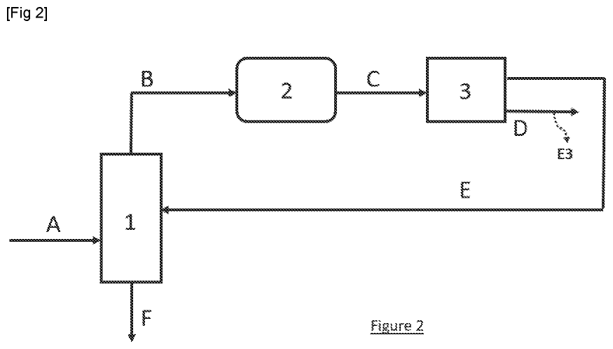

In a methyl mercaptan production unit, the vent gases E1, E2 and E3 were recovered as mentioned in FIGS. 1 and 2.

After recovery, the vent gases E1, E2 and E3 are combined and their composition is as follows:

TABLE 1

| | Amount (weight % relative to the total |
|---|---|
| Component | weight of the vent gases) |
| H₂S | 74.5 |
| MeSH | 16.9 |
| Inert matter | 8.2 |
| Water | 0.3 |
| Sulfur byproducts (DMS and DMDS) | 0.1 |
| TOTAL | 100 |

For 100 tonnes/day of methyl mercaptan produced, 3 tonnes/day of these vent gases are produced and must be incinerated.

Thus, for a unit producing 100 000 tonnes/year of methyl mercaptan, these vent gases can represent up to 3000 tonnes/year to be incinerated. Their incineration leads to 5000 tonnes/year of SO₂ emissions.

### Example 2: Example in Accordance with the Invention, with Gas-Liquid Extraction

The vent gases E1, E2 and E3 are recovered in the same way as for Example 1 and the composition of the three combined vent gases is the same:

TABLE 2

| | Amount (weight % relative to the total |
|---|---|
| Component | weight of the vent gases) |
| H₂S | 74.5 |
| MeSH | 16.9 |
| Inert matter | 8.2 |
| Water | 0.3 |
| Sulfur byproducts (DMS and DMDS) | 0.1 |
| TOTAL | 100 |

The combined vent gases are sent to a methanol absorber in order to perform the gas-liquid extraction according to the invention.

Said extraction is performed in a packed absorption column, the gas stream of the vent gases arriving at the bottom of the column and the liquid methanol arriving at the top of the column counter-currentwise.

The temperature is 46° C. and the pressure is 27 bar absolute.

The mass ratio of the gaseous vents to the methanol is 0.05.

The enriched methanol is recovered at the bottom of the column with the following composition:

TABLE 3

| Component | Amount (weight %) |
|---|---|
| Methanol | 95.3 |
| H₂S | 3.4 |
| MeSH | 1 |
| Inert matter | 0.14 |
| Water | 0.14 |
| Sulfur byproducts (DMS and DMDS) | 100 |

This absorption makes it possible to recover more than 99% of the H₂S, MeSH and sulfur byproducts that were destined for incineration without this absorber. The inert matter is almost not absorbed by methanol.

This enriched methanol is then mixed with fresh methanol (methanol that has not undergone the extraction step) to be sent to the reactor where the methyl mercaptan is produced from methanol and H₂S.

For a unit producing 100 000 tonnes/year of methyl mercaptan, this treatment of the vent gases makes it possible to recycle 2250 tonnes/year of H₂S and to recover an additional 500 tonnes/year of methyl mercaptan.

Furthermore, the sulfur products remaining in the vent gases are in negligible amount: there is no longer any release of SO₂.

1. A process for producing methyl mercaptan, comprising the following steps:
A) methanol is reacted with hydrogen sulfide to form a stream (M), preferably in gaseous form, comprising methyl mercaptan, unreacted H₂S and possibly sulfur byproducts;
B) optionally, said stream (M) is condensed;
C) at least one step of purification of said stream (M) is performed to obtain a stream enriched in methyl mercaptan;
D) the gaseous vents resulting from said at least one purification step are recovered, said gaseous vents comprising at least one sulfur compound, preferably H₂S;
E) a gas-liquid extraction of said at least one sulfur compound, preferably H₂S, is performed with liquid methanol so as to obtain a liquid methanol enriched in sulfur compound(s), preferably in H₂S; and
F) optionally, said enriched methanol is used as reagent for the reaction of step A).

2. The process for producing methyl mercaptan according to claim 1, in which, during step C), at least one phase separation step is performed, preferably by decantation, and/or at least one purge is performed.

3. The process for producing methyl mercaptan according to claim 1, in which the mass ratio of the gaseous vents relative to methanol in step E) is between 0.001 and 0.5, preferably between 0.005 and 0.1.

4. The process for producing methyl mercaptan according to claim 1, in which the gas-liquid extraction is performed at a temperature of between 0° C. and 80° C., for example between 5° C. and 80° C., preferably between 10° C. and 80° C., more preferentially between 20° C. and 70° C.

5. The process for producing methyl mercaptan according to claim 1, in which the gas-liquid extraction is performed at a pressure of between 4 and 60 bar absolute, preferably between 10 and 50 bar absolute.

6. The process for producing methyl mercaptan according to claim 1, in which said enriched methanol is used as reagent for the reaction of step A) as a mixture with fresh methanol.

7. The process for producing methyl mercaptan according to claim 1, in which said enriched methanol comprises between 0.1% and 20% by weight of H₂S, preferably between 1% and 10% by weight of H₂S, more preferentially between 1% and 5% by weight of H₂S, relative to the total weight of the enriched methanol.

8. The process for producing methyl mercaptan according to claim 1, in which said gaseous vents comprise H₂S, methyl mercaptan, inert compounds, possibly water and sulfur byproducts such as dimethyl sulfide and dimethyl disulfide.

9. The process for producing methyl mercaptan according to claim 1, in which the gas-liquid extraction is performed in at least one absorption column or in at least one tank, preferably with mechanical stirring.
